(19) United States
(12) Patent Application Publication
(10) Pub. No.: US 2015/0335677 A1
(43) Pub. Date: Nov. 26, 2015

(54) Argon is a mu opioid receptor antagonist

(71) Applicant: Helene Nancy David, Saint-Laurent de l'lle d'Orleans (CA)

(72) Inventor: Helene Nancy David, Saint-Laurent de l'lle d'Orleans (CA)

(21) Appl. No.: 14/717,972

(22) Filed: May 20, 2015

(30) Foreign Application Priority Data
May 21, 2014 (GB) 1409085.6

(51) Int. Cl. A61K 33/00; A61K 45/06; A61K 9/00

(52) U.S. Cl. CPC A61K 33/00; A61K 9/007; A61K 45/06

(57) ABSTRACT

A method of using Argon gas for treating a condition associated with mu opioid receptors and/or the vesicular monoamine transporter activity in a mammal, said method comprising the steps of: a. administering a predetermined concentration of said Argon gas in order to reduce the activity of said mu receptor and of said vesicular monoamine transporter in said mammal.

This application claims priority based on request GB1409085.6 filed 21/05/2014

### FIELD OF THE INVENTION

The present invention relates generally to drug therapy but more particularly to the use of argon as a mu opioid receptor antagonist.

### BACKGROUND OF THE INVENTION

The mu receptor is a major subclass of the opioid receptors. The mu receptor exists either at the pre- and/or post-synaptic level depending upon the body regions and cell types where it is expressed. In the brain, the mu receptor is highly expressed in various brain regions and areas such as the cortex, the thalamus, the olfactory bulb, the amygdala, the nucleus accumbens, the striatal complex including the caudate nucleus and the putamen, the solitary tract nuclei, the rostral ventromedial medulla, and the periaqueductal gray region. The mu receptor is also highly expressed in other body regions such as the spinal cord, the peripheral sensory neurons, and the intestinal tract.

Activation of the mu receptor is known to be instrumental in various diseases, but as many pharmaceutically active compounds, mu receptor antagonists produce side effects. By way of example, the well-known mu receptor antagonist naltrexone may cause liver damage. Because of this, it carries an FDA boxed warning for this side effect and its use by persons with acute hepatitis or liver failure. By another way of example, the other well-known mu receptor antagonist naloxone may cause irregular heartbeats, chest pain, short breathing, wheezing, dry cough, severe nausea or vomiting, severe headache, agitation, and confusion.

### SUMMARY OF THE INVENTION

In view of the foregoing disadvantages inherent in the known devices now present in the prior art, the present invention, which will be described subsequently in greater detail, is to provide objects and advantages which are:

To provide a mu receptor antagonist with additional inhibitory action at the vesicular monoamine transporter inhibitor, and its use in the treatment of pathological conditions in a mammal in need thereof.

In order to do so, the invention consists in the use of argon as a mu receptor antagonist with additional inhibitory action at the vesicular monoamine transporter, and with no or minimal side effects, for general pharmaceutical use.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended heret

### BRIEF DESCRIPTION OF THE DRAWINGS

FIG. 1 Inhibition of the binding of DAMGO, a mu opioid receptor agonist, by argon in membrane protein preparations. Compared to 100 vol % nitrogen (controls), 100 vol % argon altered the binding of DAMGO by decreasing Bₘₐₓ (number of receptors) by 15% and producing a trend toward reduction of 1/K_{d} (affinity), conditions that resulted in a 60% decrease in mu receptor activity (Bₘₐₓ×1/k_{d}). These data demonstrate that argon has antagonistic properties at the mu opioid receptor. *P<0.05.

FIG. 2 Effects of argon on the increase in carrier-mediated dopamine release and the reduction in KCl-evoked dopamine release induced by amphetamine in brain slices taken from the rat nucleus accumbens. (A) Experimental recording of the effect of amphetamine on carrier-mediated and KCl-evoked dopamine release. (B) The addition of amphetamine in the presence of air (control experiments) resulted in an increase in carrier-mediated dopamine release (b) compared to sham slices treated with saline solution and air (a). The addition of argon instead of air in the amphetamine solution led to a reduction of the facilitating action of amphetamine (c) as compared to control experiments performed with amphetamine and air (b). (C) The addition of amphetamine in the presence of air (control experiments) resulted in a decrease of Peak 3 (P3) KCl-evoked dopamine release (b) as compared to sham slices treated with saline solution and air (a). The addition of argon instead of air in the amphetamine solution led to an increase of the inhibiting action of amphetamine on KCl-evoked dopamine release (c) as compared to control experiments performed with amphetamine and air (b). (B-C) These data taken together indicate that argon has inhibitory properties at the type 2 vesicular monoamine transporter (see main text above). *P<0.02.

FIG. 3 Effect of argon on locomotor activity in spontaneously hypertensive rats, known to be a model for the attention deficit and hyperactivity disorder (ADHD). Spontaneously hypertensive rats treated with argon (Ar) had a lower locomotor activity than rats treated with medical air (Air). This indicates that argon reduces locomotor hyperactivity in spontaneously hypertensive rats. *P<0.02.

FIG. 4 Effects of argon on stress-induced behaviors in Sprague-Dawley rats. (A). Rats treated with argon (Ar) had a reduced number of righting reflex when handled abdomen side up compared to control animals treated with medical air (Air). (B) Rats treated with argon (Ar) had an increased number of social interactions compared to control animals treated with medical air (Air). Alternatively, in the second series of experiments, rats treated with argon (Ar) penetrated the central area of the open field to a greater extent than did control animals treated with medical air (Air), thereby indicating that argon-treated rats had reduced level of fear and anxiety. These data demonstrate that argon decreases the stress and anxiety responses induced by stressful conditions. *P<0.05.

### DETAILED DESCRIPTION

The term ‘antagonist’ and ‘inhibitory action’ are used in their normal sense in the art, i.e. a chemical compound that reduces the activity of a protein triggering a response.

Argon equilibrates rapidly within the brain by diffusing across the blood brain barrier. Argon at a pressure of around 15 atmospheres absolute acts as an agonist of the type A γ-aminobutyric acid (GABA) and benzodiazepine receptors (Abraini J. H. et al., Anesth. Analg., 2003). However, because these experiments were performed at unusual elevated concentrations, it remains uncertain whether argon would exhibit similar pharmacological properties at normal atmospheric pressure. Instead, here we show that argon at normal atmospheric pressure is a mu opioid receptor antagonist with additional inhibitory action at the vesicular monoamine transporter. Since the mechanisms of action of argon are still unknown, these pharmacological properties could likely be instrumental in the beneficial effects of this inert gas in animal models of heart attack, traumatic brain injury, acute ischemic stroke, and sensitization (addiction) to psychostimulant drugs such as amphetamine and its derivatives [Pagel P. S. et al., Anesth. Analg., 2007; Jawad N. et al., Neurosci. Lett., 2009; Loetscher P. D. et al., Crit. Care, 2009; David H. N. et al., PlosOne, 2012; Zhuang L. et al., Crit. Care Med., 2012; Brücken A. et al., Brit. J. Anaesth., 2013; David H. N. et al., Med. Gas. Res. 2014, Transl. Psych., 2015].

Unlike many other agents with antagonistic properties at the mu opioid receptor, argon is rapidly eliminated from the body through respiration, is chemically and metabolically inert, and so far as today has no reported adverse side effects. Thus, argon is widely used in humans as a carrier in diagnostic procedures (Burch et al., Nucl. Med. Commun., 1993). In addition, breathing argon at normobaric and hyperbaric pressures of 1 to 4 atmospheres absolute (equivalent to up to approximately 400%) has been reported to produce no or minimal side effect [Ackles K. N. and Fowler B., Aerosp. Med., 1971; Fowler B. and Ackles K. N., Aerosp. Med., 1972; Horrigan D. J. et al., Aviat., Space, Environ. Med., 1979; Imbert J. P. et al., Proceedings of the European Underwater and Baromedical Society, 1989].

In the preferred embodiment, the invention relates to the use of argon for general pharmaceutical use, to the use of argon for reducing the activity of the mu receptor and vesicular monoamine transporter in a mammal by administering to the mammal a therapeutically allopathic or homeopathic efficient concentration of argon.

Preferably, argon is administered in combination with a pharmaceutically acceptable carrier, diluent, or excipient. By way of example, in the pharmaceutical compositions of the present invention, argon may be admixed with any suitable binder(s), lubricant(s), suspending agent(s), carrying agents(s), containing agent(s), coating agent(s), solubilizing agent(s), selected with regard to intended route of administration and standard pharmaceutical use and medical practice.

Argon may also be administered before, after, or simultaneously with another pharmaceutically active agent or a combination of pharmaceutically active agents to decrease, increase or potentiate the pharmacological effect(s) of such agent(s), and improve the mammal's treatment and general condition. The agent(s) may be any suitable pharmaceutically active compound(s), including volatile anesthetics and inert gases such as xenon, helium and nitrous oxide

Typically, the pharmaceutical composition comprising argon, alone or in combination with another pharmaceutically active agent, is delivered to the mammal by inhalation, or oral, sublingual, transmucosal, transdermal, intravenous (bolus administration and/or infusion), neuraxial (subdural, or subarachnoidal) administration, or by any other available technique, or a combination thereof.

It is to be noted that the prior art has neither disclosed nor suggested the use of argon as a mu receptor antagonist and/or a vesicular monoamine transporter inhibitor for treating the below-mentioned diseases.

In one embodiment, the invention relates to the use of argon for reducing the activity of the mu receptor and of the vesicular monoamine transporter by administering to the mammal a therapeutically effective concentration of argon for treating a pathological condition associated with the mu receptor and/or the vesicular monoamine transporter, particularly:

Stress-induced disorders, such as anxiety, nervousness, tension, jumpiness, excitability, reduced social interactions, and other responses related to previous exposure to stressful and/or traumatic conditions.

Attention Deficit and Hyperactivity Disorder (ADHD), also known as the hyperkinetic disorder in the International Statistical Classification of Diseases and Related Health Problems of the World Health Organization, by improving executive functions, such as attentional control and inhibitory control, whose impairment causes attention deficits, hyperactivity, and impulsiveness.

In another embodiment, the invention provides a pharmaceutical composition which comprises argon and a pharmaceutically acceptable carrier, excipient or diluent, wherein the improvement is using argon for manufacturing a medicament for reducing the activity of the mu receptor and of the vesicular monoamine transporter and treating a pathological condition associated with these proteins, particularly:

Stress-induced disorders, such as anxiety, nervousness, tension, jumpiness, excitability, reduced social interactions, and other responses related to previous exposure to stressful and/or traumatic conditions.

Attention Deficit and Hyperactivity Disorder.

The amount of argon employed in the pharmaceutical composition may be the minimum concentration required to achieve the desired clinical effect in human patients. Particularly, the concentration of argon administered by inhalation is between 1 vol % and 99 vol %, advantageously between 20 vol % and 80 vol %, more advantageously between 50 vol % and 80 vol %. But, it is usual for a physician to determine the actual dosage that will be more suitable for an individual patient, and the dose will vary with the response, age, weight, and other specific condition(s) of the particular patient. There can, of course, be individual instances where higher or lower doses are merited, and such are within the scope of the invention.

The pharmaceutical composition of the present invention may also be for animal administration. Thus, the composition of the present invention, or a veterinary acceptable composition thereof, is typically administered in accordance with veterinary practice and the veterinary surgeon will determine the dose and route of administration that will be most appropriate for a particular animal.

The present invention is further described by way of examples from in vitro and in vivo studies, and with reference to the accompanying figures.

### 1. In Vitro Studies

### 1.1. Mu Receptors

Methods:

Membrane preparations were obtained from whole brains of rats untreated (n=4). The brains were crushed and homogenized in TRIS-HCl 50 mM buffer. After centrifugation, the bases of the vials were suspended in the same volume of TRIS-HCl buffer (×2). When the membrane preparation was obtained, the proteins were quantified to prepare in fine a solution at 1 mg/ml. Proteins were quantified using a BCA protein assay. Then, binding studies were performed as follows: solutions to allow calculating total binding were prepared by adding 385 μL of a TRIS-HCl buffer to 330 μL of proteins and 385 μL of [³H]-DAMGO at decreasing concentrations (n=2 per dose, N=12). Solutions to allow calculating non-specific binding were prepared in the same fashion with naloxone instead of buffer. The vials containing these solutions were left open and placed in a closed chamber to allow saturating the solutions with 100 vol % nitrogen or argon. 1000 μL of each vial were placed in a 24-well plate coated with polyethylenimine. After drying, 100 μL scintillant was added to allow counting radioactivity (×3). Specific binding was obtained by subtracting non-specific binding to total binding, and Bₘₐₓ, K_{d}, and Bₘₐₓ×1/K_{d} (mu receptor activity) were calculated.

Results:

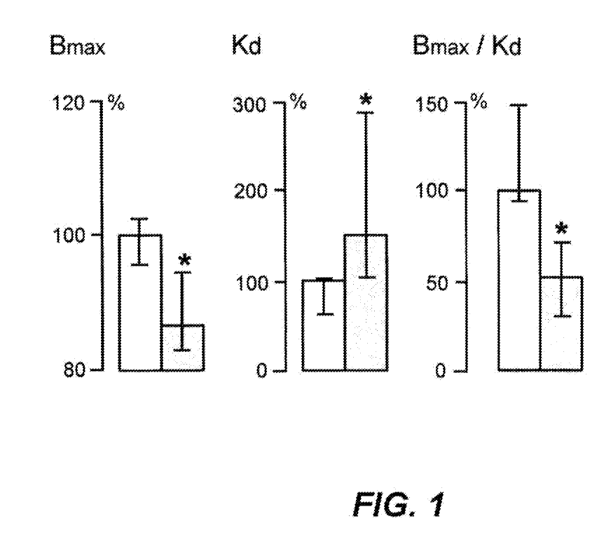

FIG. 1 shows the binding of DAMGO, a mu receptor agonist, in membrane protein preparations in the presence of 100 vol % nitrogen or 100 vol % argon. Compared to nitrogen, argon altered the binding of DAMGO by decreasing Bₘₐₓ (number of receptors) by 15% and producing a trend toward reduction of 1/K_{d} (affinity), conditions that resulted in a 60% decrease in mu receptor activity (Bₘₐₓ×1/k_{d}). These data demonstrate that argon has antagonistic properties at the mu opioid receptor. *P<0.05.

### 1.2. Type 2 Vesicular Monoamine Transporter

Methods:

Rats were killed by decapitation and the brains were carefully removed and placed in ice-cold artificial cerebrospinal fluid (aCSF). Coronal brain slices (400 μm thickness) including the nucleus accumbens (anteriority: −1.2 to +2 mm from the bregma) were cut using a tissue chopper. Before being used, brain slices (n=4 per condition) were allowed to recover at room temperature for 1 hour in oxygenated a CSF. Slices were then placed in a recording chamber (1 mL volume) at 34.5±0.5° C. and superfused at a flow rate of 1 mL/min with aCSF in the presence of amphetamine and air (nitrogen 75 vol %+oxygen 25 vol %) or argon at 75 vol % (with the remainder being oxygen). Control slices were treated with saline solution and air. Carrier-mediated and depolarization-dependent (KCl: 100 mM) dopamine release in the nucleus accumbens were monitored using a polarograph and standard glass-encased nafion-precoated carbon fiber electrodes (David H. N. et al., Biol. Psych., 2006). For each experimental condition (saline+air, amphetamine+air, amphetamine+argon), changes in dopamine release were calculated using each slice as its own control as illustrated in FIG. 1A: changes in carrier-mediated dopamine release were calculated as [B2−B1], [B3−B1], and [B4−B1]; Peak 2 (P2) and Peak 3 (P3) KCl-evoked dopamine responses were calculated as a percentage change from Peak 1 (P1) KCl-evoked dopamine release taken as a 100% value. Therefore, in the experiments with argon, argon was only administered after Peak 1 (P1) KCl-evoked dopamine release had returned to baseline (B2).

Results:

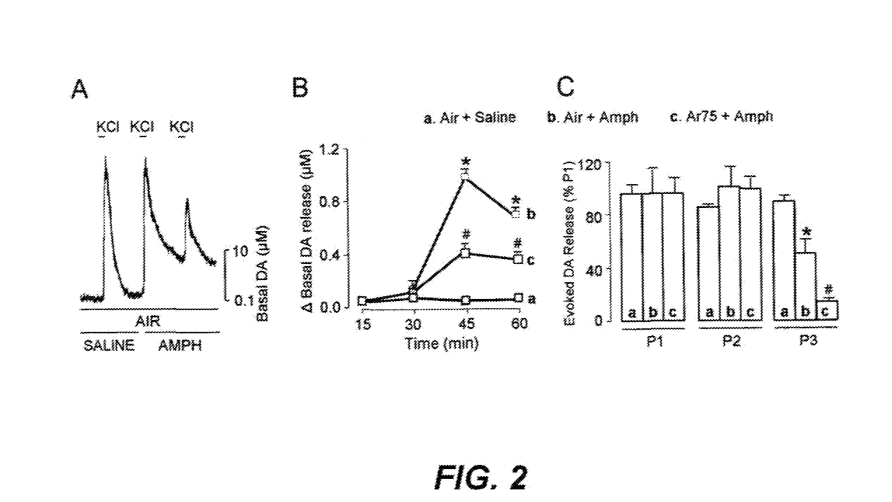

FIG. 2 shows the effects of argon on the amphetamine-induced increase in carrier-mediated dopamine release and the reduction in KCl-evoked dopamine release induced by amphetamine (experimental recording; FIG. 2A). Amphetamine acts by reversing both the dopamine transporter and the type 2 vesicular monoamine transporter. Blocking the dopamine transporter with specific inhibitors reduces the amphetamine-induced increase in carrier-mediated dopamine release but also restores the reduction in evoked dopamine release induced by amphetamine (Patel J. et al., J. Neurochem., 2003). In contrast, argon decreases the facilitating action of amphetamine on carrier-mediated dopamine release (FIG. 1B) and further potentiates the reduction in Peak 3 (P3) KCl-evoked dopamine release induced by amphetamine (FIG. 1C), effects known to result specifically from an inhibition of the type 2 vesicular monoamine transporter (Wilhelm C. J. et al., J. Exp. Pharmacol. Ther., 2004; Wilhelm C. J. et al., Biochem. Pharmacol., 2008). The lack of effect of amphetamine in the presence of air or argon (and therefore of argon) on Peak 2 dopamine release is known to be due to the fact that amphetamine yet has not reached its site of pharmacological action (David et al. Biol. Psychiatry, 2006). Taken together these data indicate that argon has inhibitory properties at the type 2 vesicular monoamine transporter. *P<0.02.

### 2. In Vivo Studies

### 2.1. Locomotor Studies

Methods:

Male spontaneously hypertensive rats (n=7-8 per group), known to show spontaneous locomotor hyperactivity and used as a model for the attention deficit and hyperactivity disorder (ADHD), were treated daily from day 1 to day 3 for 3 h with ‘medical’ air (composed of 75 vol % nitrogen and 25 vol % oxygen) or argon at 75 vol % (with the remainder being oxygen) at a flow rate of 5 L/min in a closed chamber. On day 7, rats were habituated to the activity boxes for 1 h before being recorded for locomotor activity for 1 h 30 min as detailed previously (David et al., Neuropharmacol., 2004).

Results:

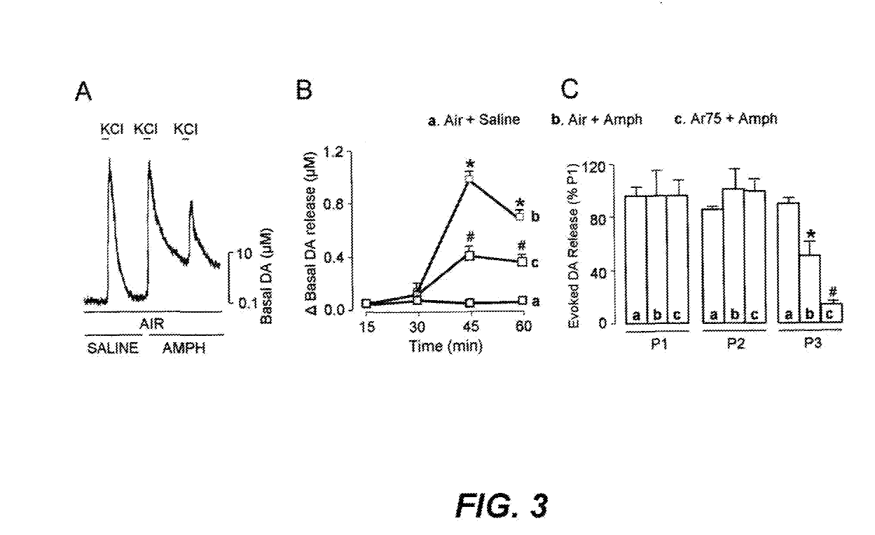

FIG. 3 illustrates the basal locomotor response of spontaneously hypertensive rats treated with air (controls) or argon. As recorded on day 7, rats treated with argon from day 1 to day 3 had a lower locomotor activity than rats treated with air. This indicates that argon reduces locomotor hyperactivity in spontaneously hypertensive rats, known to be a model for the attention deficit and hyperactivity disorder (ADHD). *P<0.02.

### 2.2. Stress-Induced Behavior Studies

Methods:

In a first series of experiments, male adult Sprague-Dawley rats (n=6 per group) were treated for 3 h from day 1 to day 3 with ‘medical’ air (composed of 75 vol % nitrogen and 25 vol % oxygen) or argon at 75 vol % (with the remainder being oxygen) at a flow rate of 5 L/min in a closed chamber. None of the animals were habituated to handling with their abdomen side up. Immediately after being treated with either air or argon from day 1 to day 3, and on day 7, rats were handled by an experimenter blind of the rats' gas treatment, with their back placed in the palm of the experimenter and their abdomen side up. The rat's stress level was rated both by the experimenter and an additional observer, also blind of the rats' gas treatment, by counting the number of righting reflex of the animals during a 1-min period on a scale of 0 to 3 with 0 being a total absence of righting reflex and 3 being repetitive righting reflexes. Then, the rats' social interaction was also evaluated. The animals were placed by group of 2 in an open field measuring 90 cm×90 cm for a 10-min period, and the time spent by the animals at a distance comprised between 2 and 5 cm was recorded and taken as a behavioral marker of social interaction.

In a second series of experiments, from day 1 to day 3, additional male Sprague-Dawley rats (n=6 per group) were placed for 10 min in an open field measuring 90 cm×90 cm, which central area measuring 50 cm×50 cm was equipped for delivering an electric shock to the animals that stayed more than 5 s in the area. From day 4 to day 6, the animals were treated for 3 h with medical air or argon at 75 vol % at a flow rate of 5 L/min in a closed chamber. Immediately after being treated with either air or argon from day 4 to day 6, and on day 8, rats were placed in the open field, and recorded during a 10-min period for the time that they spent in the central area whose electrical circuit was switched off.

Results:

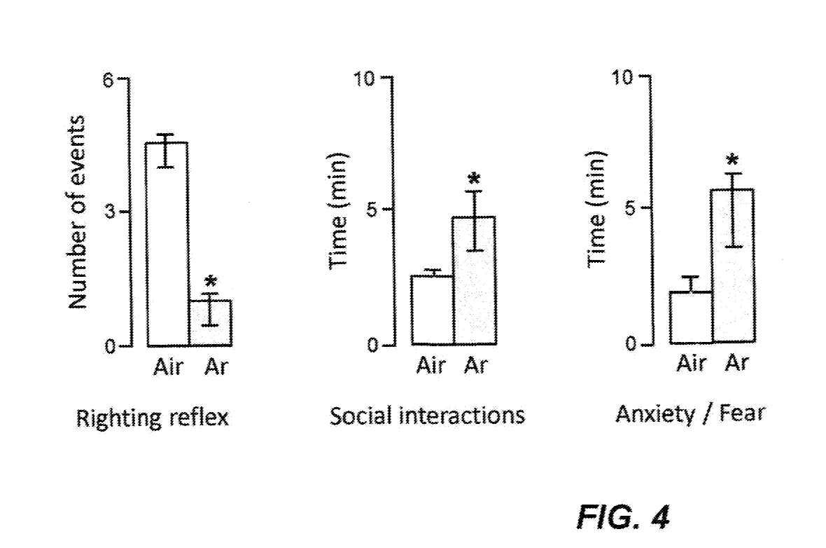

FIG. 4 shows the effects of argon on stress-induced behaviors. In the first series of experiments, rats treated with argon had a reduced number of righting reflex when handled abdomen side up (FIG. 4A) and further showed an increased number of social interactions compared to control animals treated with air (FIG. 4B) as recorded immediately after treatment on days 1 to 3, but also on day 7. Alternatively, in the second series of experiments, rats treated with argon penetrated the central area of the open field to a greater extent than did control animals as recorded immediately after treatment on days 4 to 6, but also on day 8 (FIG. 4C). These data taken together demonstrated that argon decreases the stress and anxiety responses induced by stressful conditions, and further improves social interactions. *P<0.05.

### 2.3. Conclusions

These data show that argon is a mu opioid receptor (FIG. 1) and vesicular monoamine transporter inhibitor (FIG. 2) that allows reducing both spontaneous hyperactivity in an animal model of ADHD (FIG. 3) and the behavioral responses to stressful conditions (FIG. 4). Therefore, the claims for such discoveries and indications are described hereinbelow.

As to a further discussion of the manner of usage and operation of the present invention, the same should be apparent from the above description. Accordingly, no further discussion relating to the manner of usage and operation will be provided.

1. The use of Argon gas for treating a pathological condition associated with the mu opioid receptor and/or the vesicular monoamine transporter activity in a mammal, said method comprising the steps of:
a. administering a predetermined concentration of said Argon gas in order to reduce the activity of said mu receptor and of said vesicular monoamine transporter in said mammal.

2. The use of Argon gas of claim 1, further comprising the step of:
b. administering said Argon gas in combination with a pharmaceutically acceptable carrier, diluent, or excipient.

3. The use of Argon gas of claim 2, wherein said carrier, diluent or excipient includes oxygen.

4. The use of Argon gas of claim 1, further comprising the step of:
c. administering said Argon gas before, after, or simultaneously with at least one pharmaceutically active agent.

5. The use of Argon gas of claim 4, wherein said at least one pharmaceutically active agent includes an inert gas.

6. The use of Argon gas of claim 4, wherein said at least one pharmaceutically active agent includes a combination of inert gases.

7. The use of Argon gas of claim 2 or 4, further comprising the step of:
d. delivering the pharmaceutical composition comprising said Argon gas to a mammal by inhalation, or oral, sublingual, transmucosal, transdermal, intravenous, or neuraxial administration, or any other available technique, or a combination thereof.

8. The use of Argon gas of claim 7, wherein said Argon gas combination is delivered by inhalation and is in a volume proportion of between 1% and 99% Argon gas.

9. The use of Argon gas of claim 7, wherein said Argon gas combination is delivered by inhalation and is in a volume proportion of between 20% and 80% Argon gas.

10. The use of Argon gas of claim 7, wherein said Argon gas combination is delivered by inhalation and is in a volume proportion between 50% and 80% Argon gas.

11. The use of Argon gas of claim 1, wherein said Argon gas is used for treating stress-induced disorders such as anxiety, nervousness, tension, jumpiness, excitability, and reduced social interactions.

12. The use of Argon gas of claim 1 wherein said Argon gas is used for treating at least one symptom of the Attention Deficit and Hyperactivity Disorder (ADHD).
